(19) United States
(12) Patent Application Publication
(10) Pub. No.: US 2013/0108475 A1
(43) Pub. Date: May 2, 2013

(54) Actuator-Driven Pinch Pump

(71) Applicant: Viking AT, LLC, Sarasota, FL (US)

(72) Inventor: Jeffery B. Moler, Sarasota, FL (US)

(73) Assignee: Viking AT, LLC, Sarasota, FL (US)

(21) Appl. No.: 13/661,452

(22) Filed: Oct. 26, 2012

Related U.S. Application Data
(60) Provisional application No. 61/551,530, filed on Oct. 26, 2011.

(51) Int. Cl. F04B 43/09; A61M 1/10; F04B 17/03

(52) U.S. Cl. 417/43; 417/412; 417/53; 600/16

(57) ABSTRACT

An actuator-driven pump having a resilient chamber and a plurality of smart material actuators arranged such that activation of each smart material actuator will cause the smart material actuator to compress the resilient chamber is disclosed. A controller is connected to the smart material actuators for controlling the activation and deactivation of said smart material actuators in a pattern to urge a material through the resilient chamber. Methods of pumping material through activating actuators in an actuator driven pump in a predetermined pattern are also disclosed.

### CROSS-REFERENCE TO RELATED APPLICATIONS

This application claims priority from U.S. provisional patent application Ser. No. 61/551,530 filed Oct. 26, 2011, which is herein incorporated by reference in its entirety.

### BACKGROUND

The description herein relates to a pump apparatus driven by a plurality of actuators acting on a resilient chamber such as the type of tubing often used in healthcare environments or flexible rubber or plastic tubing of the type often used in industrial environments. By compressing, or pinching, the resilient chamber in a pattern, material may be urged from one end of the chamber to another. In certain examples, the flow can be reversed by reversing the pattern. In certain examples the flow can be increased or decreased by varying the speed of the pattern or the stroke of the actuators.

Smart material actuators which use a material that will expand or contract in connection with the application of an electric potential may be used in such pumps. Where the necessary level of compression, or pinching, is within the range of the expansion range of the smart material device, such actuators may be of a design in which the smart material device acts directly upon the chamber. Where additional stroke length is required, mechanically amplified smart material actuators may be utilized, including mechanically amplified smart material actuators such as those described herein and in the incorporated references. Use of smart material actuators in certain examples of the pumps described herein can have the benefit of low power usage, fast operation, and improved controllability.

Actuator-driven pumps, including certain of the examples described herein, can be suitable in a variety of applications, including without limitation, medical applications in which it is desirable to be able to easily replace the resilient chamber with a new chamber between uses without the need to sterilize a previously used chamber. Pumps according to the examples described herein can also be useful in applications that require more precise flow control, reversibility, or low power consumption.

This application hereby incorporates by reference U.S. applications Ser. No. 11/156,408 (Apparatus and Process for Optimizing Work from a Smart Material Actuator Product), Ser. No. 13/203,737 (Mountable Arm Smart Material Actuator and Energy Harvesting Apparatus), Ser. No. 13/203,729 (Small Scale Smart Material Actuator and Energy Harvesting Apparatus, Ser. No. 13/203,743 (Smart Material Actuator Adapted for Resonant Operation); International Publications WO2012/118548 (High Speed Smart Material Actuator with Second Stage), WO 2012/079012 (Multiple Arm Smart Material Actuator with Second Stage), WO 2011/103328 (Smart Material Actuator with Enclosed Compensator); and U.S. Pat. Nos. 6,717,332 6,717,332 (Apparatus Having A Support Structure And Actuator); 6,548,938 (Apparatus Having A Pair Of Opposing Surfaces Driven By A Piezoelectric Actuator); U.S. Pat. No. 6,737,788 (Apparatus Having A Pair Of Opposing Surfaces Driven By A Piezoelectric Actuator); U.S. Pat. No. 6,836,056 (Linear Motor Having Piezo Actuators); U.S. Pat. No. 6,879,087 (Apparatus For Moving A Pair Of Opposing Surfaces In Response To An Electrical Activation); U.S. Pat. No. 6,759,790 (Apparatus For Moving Folded-Back Arms Having A Pair Of Opposing Surfaces In Response To An Electrical Activation); U.S. Pat. No. 7,132,781 (Temperature Compensating Insert For A Mechanically Leveraged Smart Material Actuator); U.S. Pat. No. 7,126,259 (Integral Thermal Compensation For An Electro-Mechanical Actuator); U.S. Pat. No. 6,870,305 (Apparatus For Moving A Pair Of Opposing Surfaces In Response To An Electrical Activation); U.S. Pat. No. 6,975,061(Apparatus For Moving A Pair Of Opposing Surfaces In Response To An Electrical Activation); U.S. Pat. No. 7,368,856 (Apparatus And Process For Optimizing Work From A Smart Material Actuator Product); and U.S. Pat. No. 6,924,586 (Uni-Body Piezoelectric Motor).

### BRIEF DESCRIPTION OF THE DRAWINGS

Other features will become apparent from the attached drawings, which illustrate certain examples of the apparatus of this invention, wherein

FIG. 1 is a perspective view of an actuator driven pump comprising three smart material actuators;

FIG. 2 is a perspective view of an actuator driven pump comprising four smart material actuators;

FIG. 3 is a perspective view of a mechanically amplified smart material actuator having a mountable actuating arm, detachable open compensator, and detachable mechanical webs, and being suitable for use in actuator driven pumps as described herein;

FIG. 4 is a side view of an alternative mechanically amplified smart material actuator suitable for use in actuator driven pumps as described herein;

FIG. 5 is a perspective view of a mechanically amplified smart material actuator having a second stage, two mountable actuating arms, a detachable open compensator, and detachable mechanical webs, and being suitable for use in actuator driven pumps as described herein; and

FIG. 6 is a perspective view of a mechanically amplified smart material actuator having a second stage, four mountable actuating arms, a detachable closed compensator, and detachable mechanical webs, and being suitable for use in actuator driven pumps as described herein.

### DESCRIPTION

While the following describes certain examples of apparatuses according to the inventions set forth in the claims, it is to be understood that this description is to be considered only as illustrative of the principles and is not to be limitative of the inventions set forth in the claims as numerous other variations, all within the scope of the claims, will readily occur to others in light of this description.

Herein, the term “adapted” shall mean sized, shaped, configured, dimensioned, oriented and arranged as appropriate.

As discussed herein, the term “smart material” refers to piezoelectric materials that change their shape upon application of an appropriate electrical potential, also including versions of such materials sometimes created by doping known piezoelectric materials to change their electrical or mechanical properties. A “smart material device” is a device comprising smart material that is adapted for a particular application. One example of a smart material device is a multilayer piezoelectric stack comprising sections of smart material, each having an opposing positive electrode and negative electrode such that the sections of the stack are electrically joined. Upon application of a suitable electric potential, the smart material sections expand. Alternatively, upon compression of the piezoelectric stack an electric potential is created between the electrodes. By stacking multiple layers together, the expansion and electrical harvesting characteristics are added together. An example of such a multilayer piezoelectric stack is a co-fired, multilayer ceramic piezoelectric stack. Such piezoelectric stacks may be formed by printing electrodes on either end of a ceramic piezoelectric material. The layers are then stacked and fired together to create a unitary structure. Such stacks are available from a number of suppliers, including NEC. Such stacks are convenient for use in actuators having various dimensions, including very small actuators on the order of two millimeters in length, 0.5 millimeters to one millimeters in thickness and one millimeters in width. As will be understood by those of skill in the art, this is only one example and many different stack sizes can be formed using different numbers of layers, and different layer thicknesses, thereby providing for actuators suitable for a wide variety of applications.

Another type of multilayer piezoelectric stack is a stack 100 formed of sections of a single-crystal piezo material. Single crystal piezo materials can be created in a variety of configurations and are considered by some to be more efficient than co-fired ceramic materials. As such, less material may be used to generate effects comparable to larger co-fired stacks. Piezoelectric stacks formed of single-crystal piezoelectric materials may conveniently be used in even smaller embodiments of actuators, including sizes, for example, of one millimeters in length (the lengthwise axis being the axis along which the crystal predominantly expands upon application of an electric current to the crystal), and 0.3 millimeters square. Once again, it will be clear to those of ordinary skill in the art that many different sizes, including both larger and smaller sizes, may be created using such stacks. It will also be clear that different varieties of smart material devices may be preferred in different applications depending on factors including cost, durability, availability, expansion potential, and electrical efficiency. Therefore, the examples described herein are not intended to be limited by a particular type or size of smart material device, except to the extent such limitation is expressly stated.

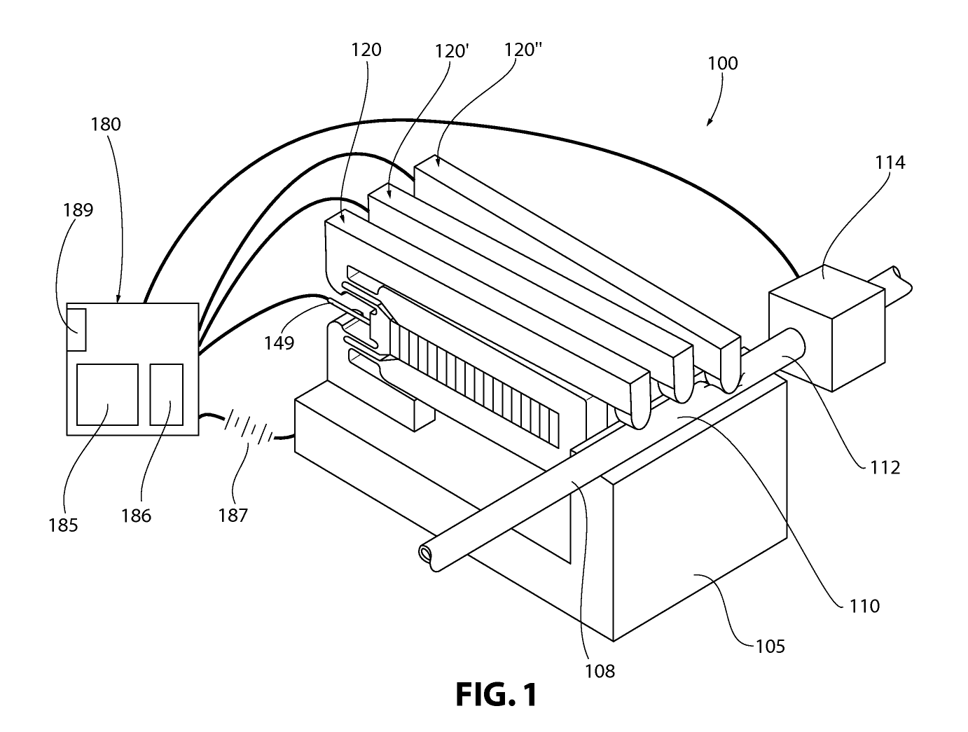

Actuator-driven pumps, including those described herein, comprise a plurality of actuators adapted to act on a resilient chamber and a controller adapted to activate such actuators according to one or more patterns. Referring to FIG. 1, actuator-driven pump 100 has three smart material actuators 120, 120′, and 120″ mounted on a base 105. Base 105 is adapted to support said resilient chamber 110 against the compression forces generated by smart material actuators 120, 120′, 120″. It will be readily understood that, while having a single base 105 is convenient, the base need not be a single structure as shown. Instead, each smart material actuator could have its own base, with each base being stackable, or allowing for larger intervals between actuators. Indeed, where the actuator itself is adapted to support resilient chamber 110, no base is needed.

Resilient chamber 110 is thus arranged in a position in which it can be acted upon by smart material actuators 120, 120′, and 120″ such that activation of smart material actuators 120, 120′, and 120″ will cause each to compress resilient chamber 110 in different locations. The compression may be such that resilient chamber 110 is compressed to a depth that may conveniently range from of one third of the diameter of the chamber, to a depth sufficient to substantially close or seal the chamber so that liquid may not pass through it. As is shown, the actuators may conveniently be arranged in a series such that the distance along the resilient chamber between each actuator is substantially the same, however, pumps according to this description also include pumps having distances between actuators that are not essentially the same.

By activating and deactivating smart material actuators 120, 120′, and 120″ according to a pattern, material (such as a gas or a liquid), can be urged from first inlet end 108 toward outlet end 112, or vice versa. It will be understood that inlet end 108 and outlet end 112 are locations on resilient chamber 110, which may be of any length and need not include separations at inlet end 108 or outlet end 112. It will be further understood that in examples in which pump 100 can operate in either direction, the terms “inlet” and “outlet” are used for convenience to illustrate operation in one direction and are not intended to imply operation in the other direction is not possible.

Smart material actuators 120, 120′, and 120″ are operably connected to controller 180. As illustrated, such connection may conveniently be electrical in nature such that controller 180 supplies the necessary electrical current to effect the required activation and deactivation of smart material actuators 120, 120′, and 120″. The power for such activation may conveniently come from a power source 187. It will be understood however, that other configurations are possible, including configurations in which smart material actuators 120, 120′, and 120″ are separately connected to a power source and controller 180 provides a digital signal to trigger the necessary activations and deactivations.

Controller 180 may conveniently comprise electrical circuitry for activating and deactivating smart material actuators 120, 120′, and 120″, that circuitry conveniently including a logic unit, a timing unit 186, and a flow adjustment unit 189. The logic unit is represented in FIG. 1 by processor 185 programmed to activate and deactivate smart material actuators in one or more repeating predetermined patterns. Processor 185 may conveniently be a microprocessor or microcontroller or any circuit or integrated circuit capable of causing smart material actuators 120, 120′, and 120″ to activate in a preprogrammed sequence. Timer 186 may conveniently be a crystal, electronic timer, or any circuit or integrated circuit capable of enabling processor 185 to identify time periods between activations of smart material actuators 120, 120′, and 120″. Processor 185 and timer 186 may be discrete components (or sets of components) or may be combined into a single component such as an ASIC or microcontroller with integrated timing capabilities. Flow adjustment unit 189 may be a rotary or slide control, a switch combined with logic circuits such that repeated activations of said switch are detected, or an electrical interface capable of conveying a digital or analog signal indicating an intended rate or rate change. In this way, it will be understood that flow adjustment unit 189 may be adapted to permit manual control of the rate of flow through resilient chamber 110, or may be an interface adapted to receive signals from an outside source.

The flexibility of controller 180 can be enhanced in certain applications, by placing a flow rate sensor 114 in operable connection with resilient chamber 110 and controller 180. As illustrated, flow rate sensor 114 is proximate to outlet end 112, but the examples described herein are not intended to be limited to such placement as other placements (including before inlet end 108 or at an upstream or downstream location remote from pump 100) are also feasible depending on the application. Processor 185 may then be programmed to selectively increase or decrease the flow rate of actuator-driven pump 100 in response to signals from flow rate sensor 114 to ensure a consistent flow or flow pattern is maintained. In this way processor 185 may adjust the operation of smart-material actuators 120, 120′, 120″ automatically based on the rate of material flow received from flow rate sensor 114, or in response to a signal from another device such as general purpose computer (not illustrated) that provides signals through flow adjustment unit 189 or directly to processor 185. Accordingly, flow direct and/or flow rate may be adjusted manually (in examples in which flow adjustment unit 185 is a manual switch), manually based on a flow rate feedback (where flow adjustment unit 185 is adapted to allow a user to set a flow rate and flow rate sensor 114 provides a signal indicating the rate of flow), automatically (in which case the flow adjustment unit 189 or processor 185 receives signals from an external device to increase or decrease speed), or automatically based on flow rate feedback (in which case flow adjustment unit 189 or processor 185 receives signals to increase or decrease flow rate and flow rate sensor 114 provides a signal indicating the rate of flow). Schematically shown controller 180 is intended to represent all of these possible variants.

Flow rate increases may be achieved by speeding up the activation pattern, including by decreasing the interval between activation of smart material actuators 120, 120′, and 120″ and flow rate decreases may be achieved by slowing down the activation pattern including by increasing the interval. Alternatively, flow rate can be selectively increased by causing controller 180 to increase the stroke length of smart material actuators 120, 120′, and 120″ thereby achieving a greater degree of compression of resilient chamber 110, or flow rate can be selectively decreased by causing controller 180 to decrease the stroke length. Likewise, flow reversal may be achieved by causing controller 180 to reverse the pattern.

A method of pumping a material utilizing an actuator-driven pump is also disclosed. As has been indicated, flow of actuator-driven pump 100 may be achieved by processor 185 causing smart material actuators 120, 120′, 120″ to activate in a predetermined sequence adapted to urge a material through resilient chamber 110. In the case of a pump comprising inlet smart material actuator 120, central smart material actuator 120′, and outlet smart material actuator 120″ the predetermined sequence may conveniently proceed as follows:

TABLE 1

| | Inlet Smart | Central | Outlet Smart |
|---|---|---|---|
| Time | Material | Smart Material | Material Actuator |
| Period | Actuator 120 | Actuator 120′ | 120″ |
| 1 | Open | Open | Activated |
| 2 | Activated | Open | Activated |
| 3 | Activated | Activated | Open |
| 4 | Activated | Activated | Activated |

As used in table 1, “Open” refers to a state in which a smart-material actuator is either not compressing resilient chamber 110 or is compressing it to a substantially lesser degree, and “Activated” refers to a state in which a smart material actuator is compressing resilient chamber 110 either completely or to a substantially greater degree. In such a progression, during time period 1, material enters the area of resilient chamber 110 between inlet smart material actuator 120 and central smart material actuator 120″. During time period 2, Inlet smart material actuator 120 activates, thereby resisting backflow and creating an initial pumping force. During time period 3, outlet smart material actuator 120″ opens and central smart material actuator 120′ activates, thereby urging the material through resilient chamber 110. During time period 4, which is optional, outlet smart material actuator 120″ activates, thereby resisting backflow. After time period 4, the cycle repeats, beginning with time period 1. It will be apparent to those of ordinary skill in the art that, during the progression, smart material actuators 120, 120′, 120″ never come into direct physical contact with the material flowing through said tube. As such, it is feasible for resilient chamber 110 to be a replaceable tube (such as are commonly used in medical applications) having a substantially sterile interior surface. Such a configuration can be desirable in certain healthcare applications as it allows avoiding contamination by enabling replacement of resilient chamber 110 (in this case tubing) between uses without requiring sterilization of the remainder of the apparatus. It is also possible for resilient chamber 110 to be a body vessel such as an artery, vein, blood vessel, or other vessel capable of conveying a bodily fluid of a human or animal, either inside or outside the body. In such applications, actuator-driven pumps of the kind described herein could be used to improve circulation and for other medical applications involving pumping of a bodily fluid. For non-medical applications, including industrial applications, resilient chamber 110 could be any type of tube or chamber capable of conveying the material to be pumped including, without limitation, plastic or rubber tubes. The material used is largely a matter of choice depending on the desired application, provided that it is capable of conveying the material to be pumped and is resilient enough to undergo repeated compression and decompression by the pump actuators as necessary for the application.

Timer 186 enables processor 185 to cause said smart material actuators 120, 120′, 120″ to activate at predetermined intervals according to such a progression, thereby causing material to be pumped. Such activations may conveniently repeat at rates between 40 Hz and 100 Hz or, depending of the stroke length of smart material actuators 120, 120′, 120″, the interior volume of resilient chamber 110, and the nature of the material to be pumped, at any other desirable rate adapted to produce the desired flow. Substantially upon adjustment of said flow control unit 189, said processor 185 will cause flow to increase or decrease by increasing or decreasing the rate. Said processor 185 may also, in conjunction with or independent of a rate increase, increase the stroke of smart material actuators 120, 120′, 120″, thereby also increasing the pumping force or flow rate. Decreasing the stroke of smart material actuators 120, 120′, 120″, would have a similar, but opposite effect of decreasing the pumping force. The combination of rate and stroke needed to obtain a given flow will vary depending on the back pressure, viscosity of the fluid, resilience of resilient chamber 110, and similar factors. The greater the viscosity of the material and/or back pressure, the greater the need for a full compression of resilient chamber 110 and a slower rate of flow. The lower the viscosity and/or back pressure, the less the need for full compression, and the higher tolerance for a faster rate. The sequence can be adjusted via electronic control to reverse the direction of flow by reversing the pattern as well. Actuator-driven pump 100 therefore operates in both directions and can proportionally control flow and pressure.

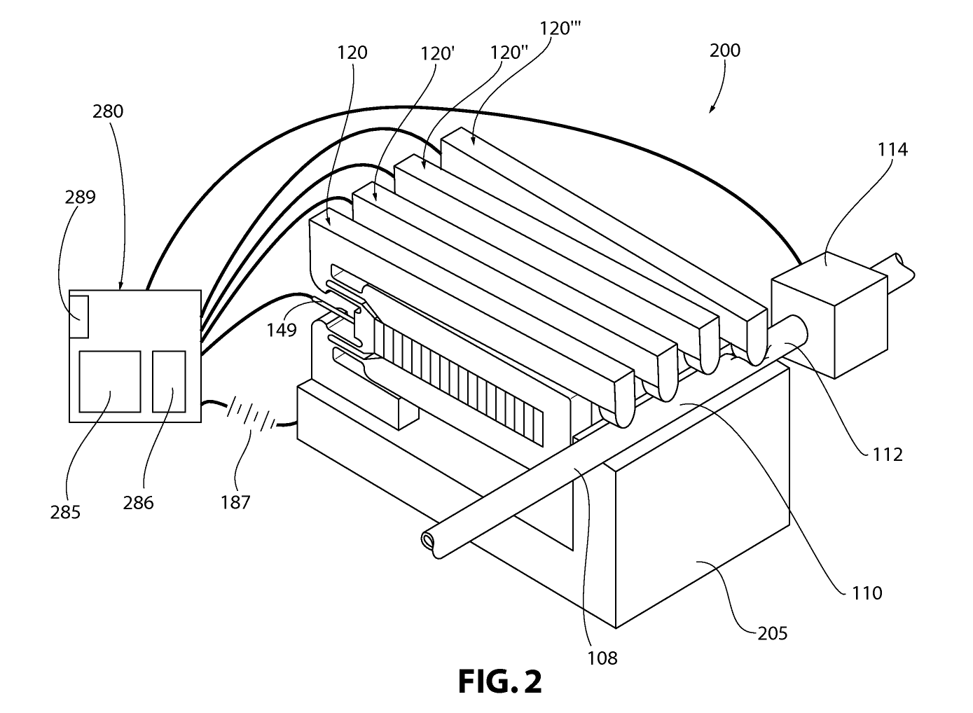

FIG. 2 illustrates an example of an actuator-driven pump 200 having four smart material actuators 120, 120′, 120″, and 120′″. As with actuator-driven pump 100 illustrated in FIG. 1, smart material actuators 120, 120′, 120″, and 120′″ are mounted on base 205, and adapted to compress resilient chamber 110 having inlet end 108 and outlet end 112, and are operably connected to controller 280. Controller 280 comprises processor 285, timer 286, and optional flow control unit 289 and is adapted to control the operation of four smart material actuators. As with the previously described example, optional flow rate sensor 114 may be operably connected to resilient chamber 110 and controller 280 in applications in which controller 280 is adapted to adjust the operation of actuator driven pump 200 based on the rate of flow. As with actuator-driven pump 100, power source 187 may conveniently be operably connected to controller 280 and smart material actuators 120, 120′, 120″, and 120′″ to provide electrical current to both, with timer 286 providing timing data and flow control unit 289 providing an interface to allow adjustment of the operation of actuator-driven pump 200 in substantially the same manner as has been previously described. In this way, it can be seen that the structure and operation of actuator-driven pump 200 is substantially similar to that of actuator-driven pump 100, except that fourth smart material actuator 120′″ is added and controller 280 and processor 285 are adapted to operate four smart material actuators instead of three.

A method of pumping a material utilizing an actuator-driven pump having four or more smart material actuators is also disclosed. As has been indicated, flow of actuator-driven pump 200 may be achieved by processor 285 causing smart material actuators 120, 120′, 120″, 120′″ to activate in a predetermined sequence adapted to urge a material through resilient chamber 110. In the case of a pump comprising inlet smart material actuator 120, first central smart material actuator 120′, second central smart material actuator 120″ and outlet smart material actuator 120′″ the predetermined sequence may conveniently proceed as follows:

TABLE 2

| | Inlet Smart | First Central | Second Central | Outlet Smart |
|---|---|---|---|---|
| Time | Material | Smart Material | Smart Material | Material |
| Period | Actuator 120 | Actuator 120′ | Actuator 120″ | Actuator 120′′′ |
| 1 | Open | Open | Open | Activated |
| 2 | Activated | Open | Open | Activated |
| 3 | Activated | Activated | Open | Activated |
| 4 | Activated | Activated | Activated | Open |
| 5 | Activated | Activated | Activated | Activated |

In such a progression, during time period 1, liquid enters the area of resilient chamber 110 between inlet smart material actuator 120 and outlet smart material actuator 120′″. During time period 2, inlet smart material actuator 120 activates, thereby resisting backflow and creating an initial pumping force. During time period 3, first central smart material actuator 120′ closes, thereby creating a further pumping force. During time period 4, second central smart material actuator 120″ closes, creating further pumping force, and outlet smart material actuator 120′″ opens allowing the pressurized material to escape. During time period 5, which should be considered optional, outlet smart material actuator 120′″ activates, thereby resisting backflow. After time period 5, the cycle repeats, beginning with time period 1.

It will be apparent to those of ordinary skill in the art in light of the foregoing description that pumps according actuator-driven pumps can also be formed with more than four actuators. Each new actuator creates an additional time period before the final time period. During that extra time period, the additional actuator will activate, ultimately leading to the opening of the final actuator. In this fashion, pumps with any number of actuators may be created. It will also be apparent in light of this description that, as additional actuators are added, it is not necessary, and indeed is not desirable, for all preceding actuators to be closed at the same time. Instead, using the four actuator progression described above as an example, once first central smart material actuator 120′ closes in time period 3, inlet smart material actuator 120 could open. Then, in time period 4, first central smart material actuator 120′ could open substantially upon second central smart material actuator 120″ closing. Using this sequence, time period 5 could be skipped, and the progression would return to time period 1 after time period 4. The following table illustrates this progression.

TABLE 3

| | Inlet Smart | First Central | Second Central | Outlet Smart |
|---|---|---|---|---|
| Time | Material | Smart Material | Smart Material | Material |
| Period | Actuator 120 | Actuator 120′ | Actuator 120″ | Actuator 120′′′ |
| 1 | Open | Open | Open | Activated |
| 2 | Activated | Open | Open | Activated |
| 3 | Open | Activated | Open | Activated |
| 4 | Open | Open | Activated | Open |

It will also be understood that the time periods discussed herein are intervals and not instants. Accordingly, depending on the back pressure, flow and fluid characteristics of the material to be pumped, during a given period, processor 185, 285 may cause one actuator to activate fully before the preceding actuator begins to open, or the preceding actuator may begin to open while the subsequent actuator is in the process of closing. Other variations are possible, and are within the scope of the present invention, and will be readily understood by those of ordinary skill in the art in light of the foregoing description.

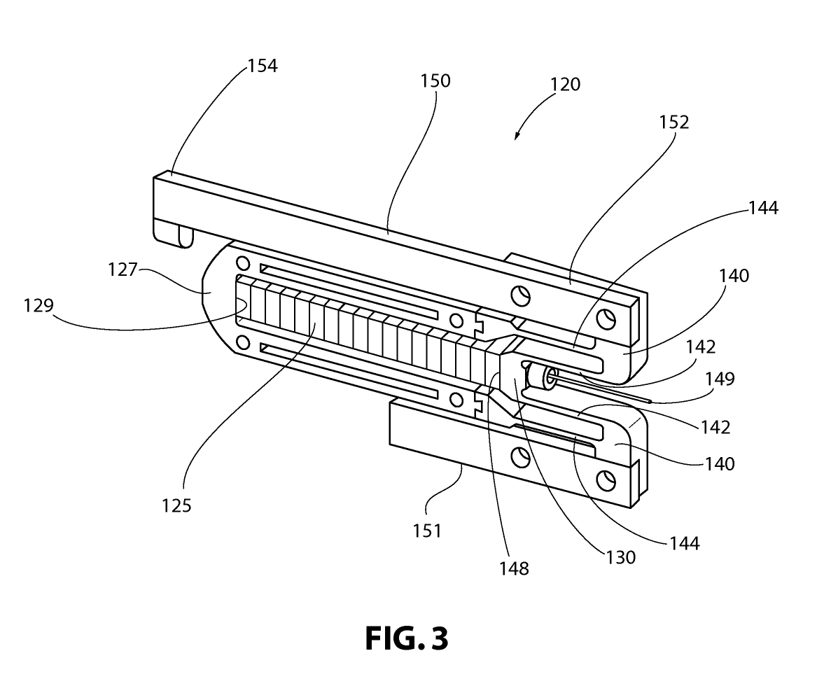

The pumps described herein may be created with any actuator adapted to compress a resilient chamber that is capable of operating at the required frequency. In particular, electro-magnetic, pneumatic and direct action piezoelectric actuators may be used. One particularly suitable type of actuator for use in the pumps described herein, however, is a mechanically amplified smart material actuator, such as those illustrated in FIGS. 3-6. FIG. 3 illustrates a mechanically amplified smart material actuator having a smart material device 125, a compensator 127, a movable supporting member 130, and two mechanical webs 140, and an actuating arm 150. Compensator 127 has a first mounting surface 129 and movable supporting member has a second mounting surface 148 that is opposed and substantially parallel to first mounting surface 129, with smart material device 125 positioned in between. Compensator 127 may be formed of a variety of materials including, without limitation steel, stainless steel, invar, aluminum, carbon fiber, and in some instances, plastics. Provided the structure of compensator 127 is suitably rigid such that stretching and flexing is kept to a practical minimum when smart material device 125 is activated.

Mechanical webs 140 may also be formed of a variety of materials including stainless steel, steel, invar, aluminum, carbon fiber and others. Mechanical webs 140 comprise inner resilient members 142 connected to movable supporting member 130 and outer resilient members 144 connected to actuating arms 150, and 151. Whereas actuating arm 151 is adapted to be connected to a pump base (such as bases 105 and 205 illustrated in FIGS. 1 and 2), actuating arm 150 is adapted to compress a resilient chamber (such as resilient chamber 110, also illustrated in FIGS. 1 and 2). Actuating arm 150 has a first actuating arm end 152 in operable connection with outer resilient member 144 and an opposed second actuating arm end 154 adapted for operable connection with a resilient chamber.

As has been described, smart material device 125 is affixed between first mounting surface 129 and second mounting surface 148. Application of an electrical potential by a controller (such as controllers 180 and 280 illustrated in FIGS. 1 and 2) to electrode 149 causes smart material device 125 to expand. The substantially parallel orientation of first mounting surface 129 and second mounting surface 148 and the restraining properties of compensator 127 are such that the expansion is substantially without angular movement. The expansion urges movable supporting member 130 away from first mounting surface 129 and causes resilient members 142, 144 to flex, thereby moving actuating arm 150 toward smart material device 125. The dimensions of mechanical webs 140 and actuating arm 150 are such that the motion of second actuating arm end 154 is across a distance greater than the expansion of smart material device 125, thereby accomplishing a mechanical amplification of the expansion of smart material device 125.

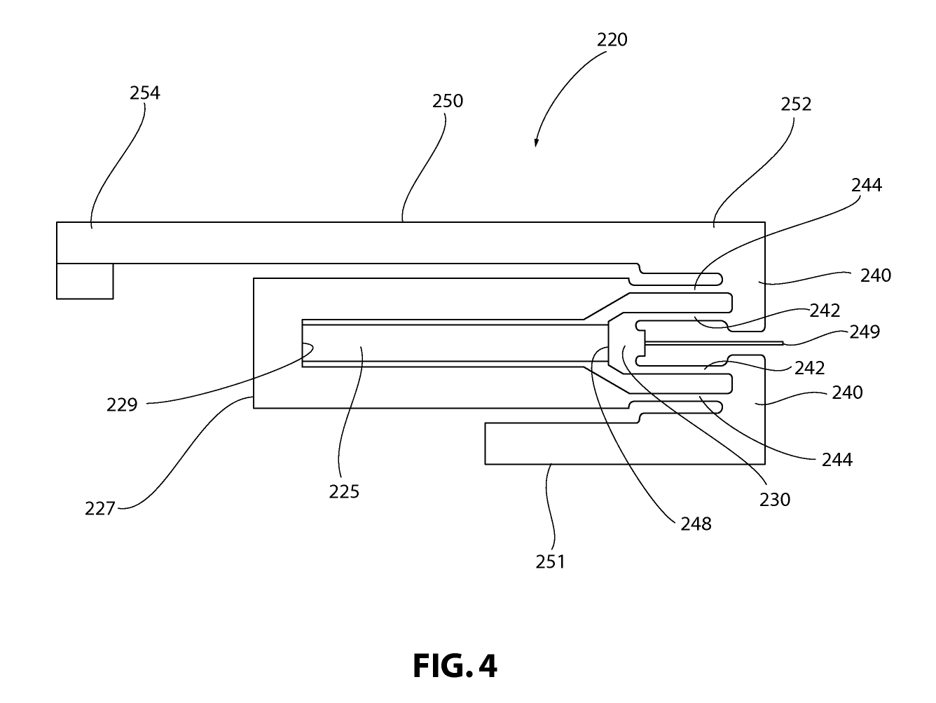

As is illustrated in FIG. 3 (and is further described in the incorporated references), compensator 127, mechanical webs 140 and actuating arms 150 and 151 may be assembled from separate parts. Such assemblies are convenient when the application calls for sufficiently large smart material actuators 120. As illustrated in FIG. 4, however, it is also possible to form them in unitary fashion, in which case much smaller smart material actuators can be formed, including smart material actuators adapted to be driven by smart material devices less than three millimeters in length. Smart material actuator 220 thus comprises a compensator 227, a movable supporting member 230, and two mechanical webs 240, and an actuating arm 250 all manufactured as single unit. As is further described in the incorporated references, such a unit may be formed from a single piece of a metal such as aluminum, steel, invar or stainless steel, or can be formed from materials such as silicon or carbon fiber.

As with the previously described example, compensator 227 has a first mounting surface 229 and movable supporting member 230 has a second mounting surface 248 that is opposed and substantially parallel to first mounting surface 229, with smart material device 225 positioned in between. In very small applications, smart material device can formed of a single crystal or layers of crystal as has been described.

Similarly, mechanical webs 240 comprise inner resilient members 242 connected to movable supporting member 230 and outer resilient members 244 connected to actuating arms 250, and 251. Whereas actuating arm 251 is adapted to be connected to a pump base (such as bases 105 and 205 illustrated in FIGS. 1 and 2), actuating arm 250 is adapted to compress a resilient chamber (such as resilient chamber 110, also illustrated in FIGS. 1 and 2). Actuating arm 250 has a first actuating arm end 252 in operable connection with outer resilient member 244 and an opposed second actuating arm end 254 adapted for in operable connection with a resilient chamber.

As has been described, smart material device 225 is affixed between first mounting surface 229 and second mounting surface 248. Application of an electrical potential by a controller (such as controllers 180 and 280 illustrated in FIGS. 1 and 2) causes smart material device 225 to expand substantially without angular movement. The expansion urges movable supporting member 230 away from first mounting surface 229 and causes resilient members 242, 244 to flex, thereby moving second actuating arm end 254 across a distance greater than the expansion of smart material device 225.

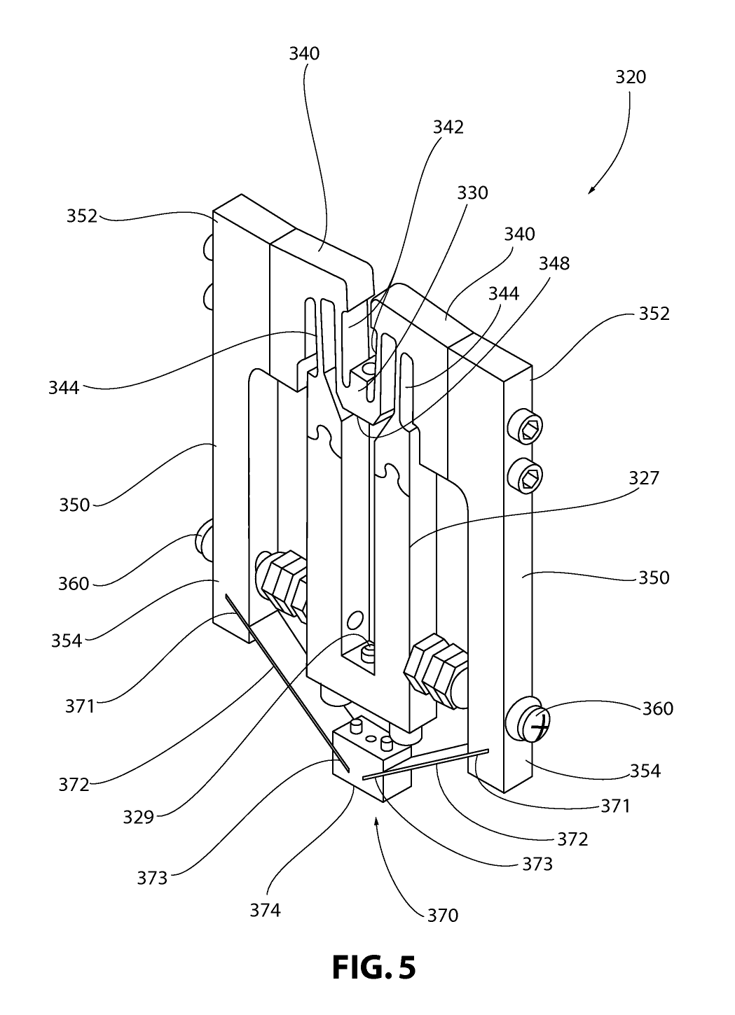

Other smart material actuator designs may also be used with the pumps described herein. One such design, illustrated in FIG. 5, is a mechanically amplified smart material actuator 320 having two actuating arms 350 and second stage 370. Similar to the previously described examples, smart material actuator 320 comprises a smart material device (not illustrated), a compensator 327, a movable supporting member 330, at least two mechanical webs 340, at least two actuating arms 350, and a second stage 370. Compensator 327 has a first mounting surface 329. Mechanical webs 340 comprise an inner resilient member 342 connected to movable supporting member 330, and an outer resilient member 344 connected to an actuating arm 350. Movable supporting member 330 comprises a second mounting surface 348 opposed and substantially parallel to first mounting surface 329. A smart material device (not illustrated) is affixed between first mounting surface 329 and said second mounting surface 348.

Actuating arms 350 comprise a first actuating arm end 352 in operable connection with one outer resilient member 344 and an opposed second actuating arm end 354. Second stage 370 comprises at least one second stage resilient member 372 having a first second stage resilient member end 371 attached to said second actuating arm end 354 and a second second stage resilient member end 373 attached to a second stage mounting block 374.

Second stage mounting block 374 can be adapted to be in operable connection with a resilient chamber such as resilient chamber 110 illustrated in FIGS. 1 and 2. Application of an electrical potential by a controller such as controllers 180, 280 illustrated in FIGS. 1 and 2 causes the smart material device (not illustrated) to expand substantially without angular movement. The expansion urges movable supporting member 330 away from first mounting surface 329 and causes said resilient members 342, 344 to flex. The flexing urges actuating arms 350 toward the smart material device (not illustrated) housed within compensator 327. That movement causes said second stage resilient members 372 to urge second stage mounting block 374 in a direction substantially parallel to the smart material device (not illustrated) and such that the motion of the second stage mounting block 374 is across a distance greater than the expansion of the smart material device (not illustrated).

As will be understood by those of ordinary skill in the art, in such applications, smart material actuator 320 can be mounted to a pump base (not illustrated) by mechanically connecting compensator 327 to the base (not illustrated) such that second stage mounting block 374 is in operable connection with a resilient chamber such as resilient chamber 110 illustrated in FIGS. 1 and 2 and can compress that resilient chamber either against a fixed surface or against an opposed actuator. Also, and as is further described in the incorporated references, optional dampeners or snubbers 360 can be used to facilitation high speed operation of smart material actuator 320 (including resonant operation where appropriate) by preventing over extension of mechanical webs 340.

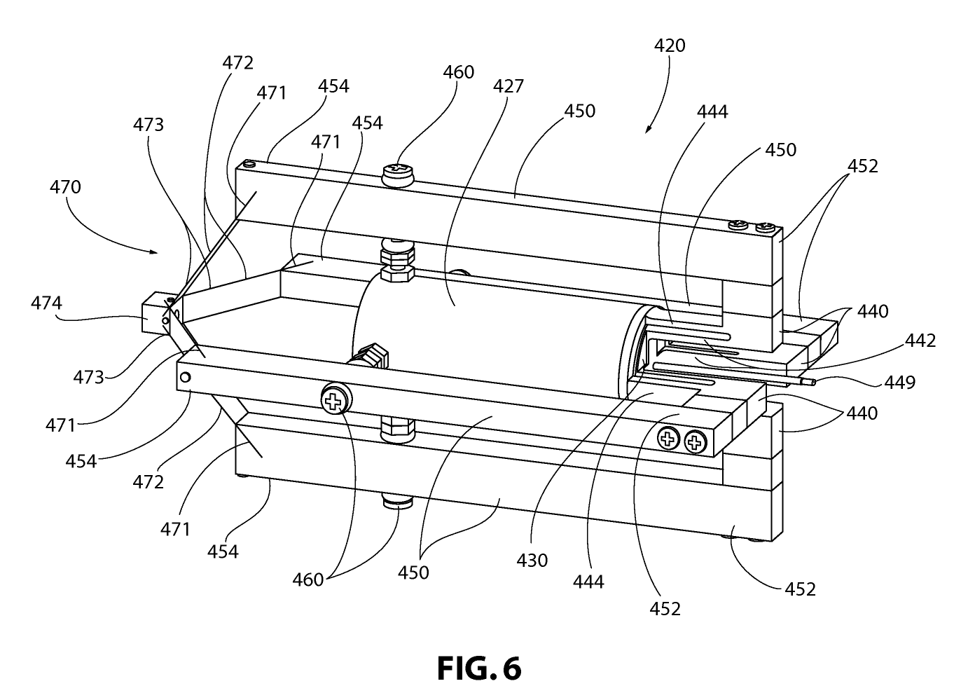

FIG. 6 illustrates another example of a mechanically amplified smart material actuator 420 having more than two actuating arms 450 and a second stage 470. Similar to the previously described example, smart material actuator 420 comprises a smart material device (not illustrated). In this example, however, a compensator 427 is an enclosed compensator that houses the smart material device (not illustrated). Movable supporting member 430 and four mechanical webs 440 enable operable connection to four actuating arms 450, which are in turn operably connected to second stage 470. Within compensator 427 is a first mounting surface (not illustrated). Mechanical webs 440 comprise inner resilient member 442 connected to movable supporting member 430, and outer resilient members 444 connected to an actuating arms 450 and compensator 427. Movable supporting member 430 comprises a second mounting surface (not illustrated) opposed and substantially parallel to the first mounting surface (not illustrated) within compensator 427 with a smart material device (not illustrated) affixed in between.

Actuating arms 450 comprise a first actuating arm end 452 in operable connection with one outer resilient member 444 and an opposed second actuating arm end 454. Second stage 470 comprises four second stage resilient members 472, each having a first second stage resilient member end 471 attached to second actuating arm end 454 and a second second stage resilient member end 473 attached to a second stage mounting block 474.

As described in connection with smart material actuator 420, second stage mounting block 473 can be adapted to be in operable connection with a resilient chamber such as resilient chamber 110 illustrated in FIGS. 1 and 2. Expansion of the smart material device (not illustrated) urges movable supporting member 430 away from compensator 427 and causes resilient members 442, 444 to flex. The flexing urges actuating arms 450 toward the smart material device (not illustrated) housed within compensator 427. That movement causes said second stage resilient members 472 to urge second stage mounting block 474 in a direction substantially parallel to the smart material device (not illustrated) and such that the motion of the second stage mounting block 474 is across a distance greater than the expansion of the smart material device (not illustrated).

Smart material actuator 420 can be mounted to a pump base (not illustrated) by mechanically connecting compensator 427 to the base (not illustrated) such that second stage mounting block 474 is in operable connection with a resilient chamber such as resilient chamber 110 illustrated in FIGS. 1 and 2 and can compress that resilient chamber either against a fixed surface or against an opposed actuator. Also, and as is further described in the incorporated references, optional dampeners or snubbers 460 can be used to facilitation high speed operation of smart material actuator 420 (including resonant operation where appropriate) by preventing over extension of mechanical webs 440.

Additional suitable smart material actuator examples, including examples having enclosed compensators, more than two actuating arms, and being capable of operation at high speeds or in a resonant condition are described in the incorporated references.

As will be readily recognized by those of ordinary skill in the art, pumps as described herein may be manufactured in a variety of sizes adapted to the needs of differing applications. Larger chambers and more viscous materials shall respond to larger and/or more powerful actuators to obtain a given flow rate. Larger resilient chambers will also require actuators with a larger stroke length to obtain the desired compression.

Other variations and embodiments of the pumps and actuators described herein will be apparent to those of ordinary skill in the art in light of this specification, all of which are within the scope of the present invention as claimed. In addition, while the scale used in the figures is illustrative of the principles of the components described herein, it will be apparent to those of skill in the art that different dimensions (including without limitation smart material device sizes, actuating arm lengths, mechanical web dimensions, and distances between actuating arms and compensators) will be applicable in different applications. Accordingly nothing in the foregoing description is intended to imply that the claimed invention is to be limited to the examples described or illustrated herein.

What is claimed is:

1. An actuator-driven pump comprising:
a resilient chamber;
a plurality of smart material actuators arranged such that activation of each smart material actuator will cause the smart material actuator to compress said resilient chamber;
a controller connected to said smart material actuators for controlling the activation and deactivation of said smart material actuators;
wherein said controller causes said smart material actuators to activate and deactivate such that the compression of said resilient chamber urges a material through said resilient chamber.

2. An actuator-driven pump according to claim 1 wherein at least one said smart material actuator is a mechanically amplified smart material actuator comprising a smart material device, a compensator, a movable supporting member, at least one mechanical web, and at least one actuating arm wherein
said compensator has a first mounting surface,
said mechanical web comprises an inner resilient member connected to said movable supporting member, and an outer resilient member;
said movable supporting member comprises a second mounting surface opposed and substantially parallel to said first mounting surface,
said actuating arm comprise a first actuating arm end in operable connection with said outer resilient member and an opposed second actuating arm end in operable connection with said resilient chamber;
said smart material device is affixed between said first mounting surface and said second mounting surface;
wherein application of an electrical potential by said controller causes said smart material device to expand substantially without angular movement, thereby urging said movable supporting member away from said first mounting surface and causing said resilient members to flex, thereby moving said actuating arm toward said smart material device such that motion of said second actuating arm end is across a distance greater than the expansion of said smart material device.

3. An actuator-driven pump according to claim 1 wherein at least one said smart material actuator is a mechanically amplified smart material actuator comprising a smart material device, a compensator, a movable supporting member, at least two mechanical webs, at least two actuating arms, and a second stage wherein
said compensator has a first mounting surface,
said mechanical webs comprise an inner resilient member connected to said movable supporting member, and an outer resilient member;
said movable supporting member comprises a second mounting surface opposed and substantially parallel to said first mounting surface,
each said actuating arm comprise a first actuating arm end in operable connection with one said outer resilient member and an opposed second actuating arm end;
said smart material device is affixed between said first mounting surface and said second mounting surface;
said second stage comprises at least one second stage resilient member having a first second stage resilient member end attached to said second actuating arm end and a second second stage resilient member end attached to a second stage mounting block in operable connection with said resilient chamber;
wherein application of an electrical potential by said controller causes said smart material device to expand substantially without angular movement, thereby urging said movable supporting member away from said first mounting surface and causing said resilient members to flex, thereby urging said actuating arms toward said smart material device, thereby causing said second stage resilient members to urge said second stage mounting block in a direction substantially parallel to said smart material device such that motion of said second stage mounting block is across a distance greater than the expansion of said smart material device.

4. An actuator-driven pump according to claim 1 having three smart material actuators.

5. An actuator-driven pump according to claim 1 having four smart material actuators.

6. An actuator-driven pump according to claim 1 having more than four smart material actuators.

7. An actuator-driven pump according to claim 1 wherein said controller comprises a processor programmed to
activate and deactivate said smart material actuators in a repeating pre-determined pattern,
to selectively increase the rate of flow of said material through said pump by increasing the speed at which such pattern repeats, and to
to selectively decrease the rate of flow of said material through said pump by decreasing the speed at which said pattern repeats.

8. An actuator-driven pump according to claim 1 wherein
said resilient chamber comprises a tube having an inlet end and an outlet end; and
said controller comprises a processor;
said controller is programmed to selectively cause said smart material actuators to activate and deactivate in a first pre-determined pattern to urge material in said chamber from said inlet end toward said outlet end or to cause said smart material actuators to activate and deactivate in a second predetermined pattern to urge material in said chamber from said outlet end toward said inlet end.

9. An actuator-driven pump according to claim 1 wherein the resilient chamber is a replaceable tube having a substantially sterile interior surface.

10. An actuator-driven pump according to claim 1 wherein the resilient chamber is a body vessel.

11. An actuator-driven pump according to claim 1 further comprising a flow rate sensor in operable connection with the resilient chamber and said controller wherein said controller comprises a processor programmed to selectively increase the flow rate of the actuator-driven pump by decreasing the interval between activation of said smart material actuators in response to a signal from said flow rate sensor.

12. An actuator-driven pump according to claim 1 further comprising a flow rate sensor in operable connection with the resilient chamber and said controller wherein said controller comprises a processor programmed to selectively increase the flow rate of the actuator-driven pump by increasing the stroke length of said smart material actuators in response to a signal from said flow rate sensor.

13. An actuator-driven pump according to claim 1 wherein at least one said smart material actuator comprises a smart material device less than three millimeters in length.

14. An actuator-driven pump according to claim 1 wherein at least one said smart material actuator comprises a smart material device consisting of a single piezoelectric crystal.

15. An actuator-driven pump according to claim 1 wherein at least one said smart material actuator comprises a smart material device consisting of a plurality of electrically connected smart material crystals arranged in a stack.

16. A method of pumping a material utilizing an actuator-driven pump comprising
a resilient chamber having an inlet end and an outlet end, and
an inlet smart material actuator proximate to said inlet end, an outlet smart material actuator proximate to said outlet end, at least one central smart material actuator positioned between said inlet smart material actuator and said outlet smart material actuator,
said method comprising causing said smart material actuators to compress and release said resilient chamber in a pattern comprising at least four time periods wherein
during a substantial portion of said first time period said inlet smart material actuator is open, said central smart material actuator is open, and said outlet smart material actuator is closed;
during a substantial portion of said second time period said inlet smart material actuator is closed, said central smart material actuator is open, and said outlet smart material actuator is closed;
during a substantial portion of said third time period said inlet smart material actuator is closed, said central smart material actuator is closed, and said outlet smart material actuator is open; and
during a substantial portion of said fourth time period said inlet smart material actuator is closed, said central smart material actuator is closed, and said outlet smart material actuator is closed.

17. A method of pumping a material utilizing an actuator-driven pump comprising
a resilient chamber having an inlet end and an outlet end, and
an inlet smart material actuator proximate to said inlet end, an outlet smart material actuator proximate to said outlet end, a first central smart material actuator proximate to said inlet smart material actuator and at least one second central smart material actuator proximate to said outlet smart material actuator,
wherein said method comprises repeatedly opening and closing said inlet smart material actuator, said first central smart material actuator, said second central smart material actuator, and said outlet smart material actuator in a predetermined pattern.

18. The method of claim 17 wherein said predetermined pattern comprises five time periods and
during a substantial portion of said first said time period said inlet smart material actuator is open, said first central smart material actuator is open, said second central smart material actuator is open, and said outlet smart material actuator is closed;
during a substantial portion of said second time period said inlet smart material actuator is closed, said first central smart material actuator is open, said second central smart material actuator is open, and said outlet smart material actuator is closed;
during a substantial portion of said third time period said inlet smart material actuator is closed, said first central smart material actuator is closed, said second central smart material actuator is open, and said outlet smart material actuator is closed;
during a substantial portion of said fourth time period said inlet smart material actuator is closed, said first central smart material actuator is closed, said second central smart material actuator is closed, and said outlet smart material actuator is open; and
during a substantial portion of said fourth time period said inlet smart material actuator is closed, said first central smart material actuator is closed, said second central smart material actuator is closed, and said outlet smart material actuator is closed.

19. The method of claim 17 wherein said predetermined pattern comprises four time periods and wherein
during a substantial portion of said first said time period said inlet smart material actuator is open, said first central smart material actuator is open, said second central smart material actuator is open, and said outlet smart material actuator is closed;
during a substantial portion of said second time period said inlet smart material actuator is closed, said first central smart material actuator is open, said second central smart material actuator is open, and said outlet smart material actuator is closed;
during a substantial portion of said third time period said inlet smart material actuator is open, said first central smart material actuator is closed, said second central smart material actuator is open, and said outlet smart material actuator is closed; and
during a substantial portion of said fourth time period said inlet smart material actuator is open, said first central smart material actuator is open, said second central smart material actuator is closed, and said outlet smart material actuator is open.
